(19) United States
(12) Patent Application Publication
(10) Pub. No.: US 2025/0116624 A1
(43) Pub. Date: Apr. 10, 2025

(54) PROCESSES FOR DETERMINING FORMATION SALINITY AND IDENTIFYING OIL BEARING ZONES IN FRESHWATER PAY ZONES

(71) Applicants: Schlumberger Technology Corporation, Sugar Land, TX (US); Saudi Arabian Oil Company, Dhahran (SA)

(72) Inventors: Mohammed Fadhel Al-Hamad, Saihat (SA); Wael Abdallah, Al-Khobar (SA); Shouxiang Mark Ma, Dhahran (SA); Mustafa A. Huwaider, Dhahran (SA)

(21) Appl. No.: 18/483,112

(22) Filed: Oct. 9, 2023

(51) Int. Cl. G01N 27/06; E21B 49/02; E21B 49/08; G01N 1/44; G01N 33/24; G01N 33/28

(52) U.S. Cl. CPC G01N 27/06; E21B 49/02; E21B 49/081; G01N 1/44; G01N 33/24; G01N 33/2823

(57) ABSTRACT

Processes for determining formation salinity and/or processes for identifying oil bearing and/or water bearing zones in freshwater or relatively low salinity formations. In some embodiments, the process for determining formation water salinity can include measuring resistivity of a formation fluid sample at a first temperature (T₁) and at a second temperature (T₂), where T₁ and T₂ can be separated by a temperature difference (ΔT). The process can also include calculating a resistivity factor value based on the resistivities measured at T₁ and T₂. The process can also include determining a salinity of the formation fluid sample based on the resistivity factor value and the ΔT. The process can also include initiating a downhole operation using the determined salinity of the formation fluid sample.

### FIELD

Some embodiments described generally relate to processes for determining formation salinity. Other embodiments described generally relate to processes for identifying oil bearing and/or water bearing zones in freshwater or relatively low salinity formations.

### BACKGROUND

Determining formation water salinity accurately downhole is of great importance for many aspects of reservoir engineering, reservoir production, and petroleum economics. Formation water salinity can be obtained by direct measurement on a collected water sample, by drill stem or wireline tests, or estimated from wireline electrical logs. Oil bearing zones can also be identified by several logging techniques such as dielectric logs and nuclear magnetic resonance (NMR) logs.

These processes, however, can suffer from several factors related to the formation or to the logging tool itself, including contamination and/or analytical error. While some contaminations can be managed in the lab and easily removed, some contaminations can be difficult to manage and, therefore, can affect the accuracy of the measurements. If the formation is drilled with an oil-based mud, for example, it is generally difficult to detect and remove the contamination. Therefore, part of the contamination can mix with the sampled formation water, and for formations with freshwater, this will result inaccurate salinity estimation. Such error in the data, if not predicted properly, can negatively affect some decisions in reservoir development and planning.

There is a need, therefore, for improved processes for determining formation salinity and/or identifying oil bearing and/or water bearing zones in freshwater or relatively low salinity formations.

### SUMMARY

Processes for determining formation salinity and/or processes for identifying oil bearing and/or water bearing zones in freshwater or relatively low salinity formations are provided. In some embodiments, a process for determining formation water salinity can include measuring resistivity of a formation fluid sample at a first temperature (T₁) and measuring resistivity of the formation fluid sample at a second temperature (T₂). T₁ and T₂ can be separated by a temperature difference (ΔT). The process can also include calculating a resistivity factor value based on the resistivity measured at T₁ and the resistivity measured at T₂. The process can also include determining a salinity of the formation fluid sample based on the resistivity factor value and the ΔT. The process can also include initiating a downhole operation based on the determined salinity of the formation fluid sample.

In some embodiments, a process for identifying oil and water bearing zones in a freshwater environment or a low salinity environment can include generating a resistivity factor value model that can include at least two modeled resistivity factor values at a first temperature (T₁) and a second temperature (T₂), respectively. T₁ and T₂ can be separated by a temperature difference (ΔT). The process can also include measuring resistivity of a formation fluid sample at T₁ and measuring resistivity of the formation fluid sample at T₂. The process can also include calculating a resistivity factor value based on the resistivity measured at T₁ and the resistivity measured at T₂. The process can also include determining whether the formation fluid sample is a water-bearing formation fluid sample or an oil-bearing formation fluid sample based on the calculated resistivity factor value by comparing the calculated resistivity factor value to a corresponding modeled resistivity factor in the resistivity factor value model. The process can also include initiating a downhole operation based on the determination of the formation fluid sample being a water-bearing formation fluid sample or an oil-bearing formation fluid sample.

### BRIEF DESCRIPTION OF THE DRAWINGS

The subject disclosure is further described in the detailed description which follows, in reference to the noted plurality of drawings by way of non-limiting examples of the subject disclosure, in which like reference numerals represent similar parts throughout the several views of the drawings.

FIG. 1 depicts a flow diagram for an illustrative process for determining formation water salinity, according to one or more embodiments described.

FIG. 2 depicts a flow diagram for an illustrative a process for identifying oil and water bearing zones in a freshwater environment or a low salinity environment, according to one or more embodiments described.

FIG. 3 depicts a schematic of an illustrative computing system that can be configured to carry out one or more steps in the flow diagrams depicted in FIGS. 1 and/or 2, according to one or more embodiments described.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions can be made to achieve certain goals, such as compliance with system-related and/or operation-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Certain examples commensurate in scope with the claimed subject matter are discussed below. These examples are not intended to limit the scope of the disclosure. Indeed, the present disclosure may encompass a variety of forms that may be similar to or different from the examples set forth below.

When introducing elements of various embodiments of the present disclosure, the articles “a,” “an,” and “the” are intended to mean that there are one or more of the elements. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features. Furthermore, the phrase A “based on” B is intended to mean that A is at least partially based on B. Moreover, unless expressly stated otherwise, the term “or” is intended to be inclusive (e.g., logical OR) and not exclusive (e.g., logical XOR). In other words, the phrase A “or” B is intended to mean A, B, or both A and B.

Additionally, certain terms are used throughout the following description and claims to refer to particular components. As one skilled in the art will appreciate, various entities can refer to the same component by different names, and as such, the naming convention for the elements described herein is not intended to limit the scope of the invention, unless otherwise specifically defined herein. Further, the naming convention used herein is not intended to distinguish between components that differ in name but not function.

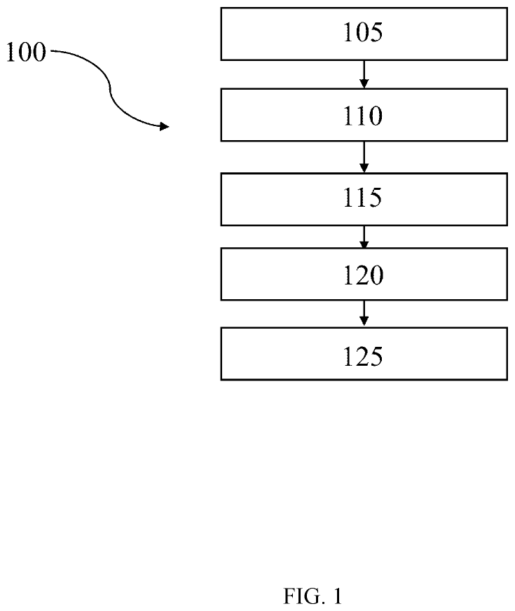

FIG. 1 depicts a flow diagram for an illustrative process 100 for determining formation water salinity, according to one or more embodiments. The process 100 can include measuring resistivity of a formation fluid sample at a first temperature (T₁) 105. The process 100 can also include measuring resistivity of the formation fluid sample at a second temperature (T₂) 110. The second temperature (T₂) can be greater than the first temperature (T₁) such that T₁ and T₂ can be separated by a temperature difference (ΔT). The process 100 can also include calculating a resistivity factor value for the formation fluid sample 115, based on the resistivities measured in steps 105 and 110. The process 100 can also include determining a salinity of the formation fluid sample 120, based on the resistivity factor value calculated in step 115 and the temperature difference (ΔT) between T₁ and T₂. The process 100 can also include initiating a downhole operation 125 using the salinity of the formation fluid sample determined in step 120. In some embodiments, the process 100 can also include measuring the resistivity of the formation fluid sample at one or more additional temperatures, e.g., a third temperature (T₃), a fourth temperature (T₄), a fifth temperature (T₅), a sixth temperature (T₆), a seventh temperature (T₇), and so on.

When the resistivity of the formation fluid sample is measured at three or more different temperatures, the temperature difference between any two sets of adjacent temperatures can be the same or different with respect to one another. For example, when the resistivity of the formation fluid sample is measured at a first temperature (T₁), a second temperature (T₂), and a third temperature (T₃), T₁ can be less than T₂, and T₂ can be less than T₃ and the temperature difference (ΔT_{1/2}) between T₁ and T₂ can be the same or different than the temperature difference (ΔT_{2/3}) between T₂ and T₃. For example, in some embodiments, the temperature difference (ΔT_{1/2}) between T₁ and T₂ can be 50° C. and the temperature difference (ΔT_{2/3}) can be 25° C. In other examples, the temperature difference (ΔT_{1/2}) between T₁ and T₂ can be 50° C. and the temperature difference (ΔT_{2/3}) can also be 50° C.

In some embodiments, the temperature difference (ΔT) between T₁ and T₂ and the temperature difference between any additional/optional temperature measurements, can independently be in a range from 1° C., 3° C., 5° C., 7° C., 10° C., 15° C., 20° C., 25° C., 30° C., 35°° C., 40° C., 45° C., 50° C., 55° C., 60°° C., 65° C., 70°° C., or 75° C. to 80° C., 85° C., 90° C., 95° C., 100° C., 105° C., 110° C., 115° C., 120° C., 125° C., 130° C., 135° C., 140° C., 145° C., 150° C., 155° C., or 160° C. In some embodiments, the temperature difference (ΔT) can be at least 1° C., at least 3°° C., at least 5° C., at least 7° C., at least 10° C., at least 15° C., at least 20°° C., at least 25° C., at least 30° C., at least 35° C., at least 40°° C., at least 45°° C., at least 50° C., at least 55° C., at least 60° C., at least 65° C., at least 70° C., or at least 75° C.

In some embodiments, the resistivity of the formation fluid sample can be measured at the first temperature (T₁) and then the resistivity of the formation fluid sample at the second temperature (T₂) can be measured. In other embodiments, the resistivity of the formation fluid sample can be measured at the second temperature (T₂) and then the resistivity of the formation fluid sample can be measured at the first temperature (T₁). Preferably, the resistivity of the formation fluid sample is measured at the first temperature (T₁) and then the resistivity is measured at the second temperature (T₂) such that the formation fluid sample can be heated from the first temperature (T₁) to the second temperature (T₂) rather than cooled from the second temperature (T₂) to the first temperature (T₁).

In some embodiments, the resistivity of the formation fluid sample at the first and second temperatures (T₁ and T₂) can be measured at the surface, e.g., in a laboratory, mobile analysis unit, or anywhere else. In such embodiments, a formation fluid sample can be obtained from a downhole location using any suitable formation fluid sampling tool. Illustrative formation fluid sampling tools can include, but are not limited to, the slimline single-phase reservoir fluid sampler (SLS) tool, the single-phase multisample chamber (SPMC) tool, the SCAR® inline independent reservoir fluid sampling tool, the single-phase reservoir sampler (SRS) tool, the nonreactive reservoir sampler (NRS) tool, the MDT modular formation dynamics tester tool, all available from Schlumberger, can be used to obtain one or more formation fluid samples from a downhole location. In such embodiments, the resistivity of the formation fluid sample can be measured at two or more temperatures within a high pressure high temperature (HPHT) resistivity cell. The formation fluid sample can be heated via any suitable source, e.g., an electrical heating element or microwave radiation. In some embodiments, when the formation fluid sample is measured at the surface, the first temperature (T₁) can be about ambient temperature, e.g., about 25° C. In other embodiments, when the formation fluid sample is measured at the surface, the first temperature (T₁) can be greater than ambient temperature, e.g., about 50° C. or greater.

In some embodiments, the resistivity of the formation fluid sample at the first and second temperatures (T₁ and T₂) can be measured at a downhole location within a wellbore or within the formation the wellbore traverses. In such embodiments, any suitable downhole tool capable of measuring resistivity of a formation fluid sample at the first temperature (T₁) and the second temperature (T₂) can be used to obtain the resistivity measurements. In some embodiments, the first temperature (T₁) the resistivity of the formation fluid sample can be measured at can be the ambient downhole temperature. In other embodiments, the first temperature (T₁) the resistivity of the formation fluid sample can be measured at can be greater than the ambient downhole temperature. In some embodiments, the formation fluid sample can be heated from the ambient downhole temperature to the second temperature (T₂) or to the first temperature (T₁) and then to the second temperature (T₂). The formation fluid sample can be heated downhole via any suitable source, e.g., an electrical heating element or microwave radiation. An illustrative downhole tool that can be used to measure the resistivity of a formation fluid sample at a downhole location at two or more different temperatures can include the wellbore tool described in U.S. Pat. No. 11,578,585.

In some embodiments, the resistivity of the formation fluid sample can be measured at the plurality of temperatures while the formation fluid sample remains under the same or substantially the same pressure. The particular pressure the formation fluid sample is at when the resistivity measurements are obtained at the plurality of temperatures is not critical. For example, in some embodiments, the formation fluid sample can be at a pressure of about 3.5 MPa, about 7 MPa, about 10 MPa, or any other downhole pressure or pressure within a measurement cell at the surface such that the pressure remains substantially the same during the resistivity measurements of the formation fluid sample at the plurality of temperatures. It should be understood that the pressure can vary some when the resistivity of the formation fluid sample is measured at the plurality of temperatures, especially if the resistivity measurements are obtained for a formation fluid sample in a closed measurement cell, but such variation is acceptable. For example, in some embodiments, the pressure the formation fluid sample is at when the resistivity measurements are obtained at the plurality of temperatures can vary by about +/−1%, +/−3%, +/−5%, +/−7%, or +/−10%.

As noted above, once the resistivity of the formation fluid sample has been measured at two or more temperatures, e.g., T₁ and T₂, the process 100 can further include calculating a resistivity factor value based on the resistivity measured at T₁ and the resistivity measured at T₂ 115. The resistivity factor value can be calculated according to the following equation (I):

$$Resistivity Factor = \frac{{Resistivity}_{T 2}}{{Resistivity}_{T 1}} \qquad ( I )$$

where, Resistivity_{T2} is the resistivity of the formation fluid sample at the second temperature (T₂), Resistivity_{T2} is the resistivity of the formation fluid sample at the first temperature (T₁), and T₂ is greater than T₁. As also noted above, the process 100 can also include determining the temperature difference (ΔT) between the first and second temperatures (T₁ and T₂).

In some embodiments, as further described below, when the resistivity of the formation fluid sample is measured at three or more temperatures, e.g., T₁, T₂, T₃, a resistivity factor value for the formation fluid sample at T₂ and T₃ can be determined by dividing the resistivity of the formation fluid sample at T₂ by the resistivity of the formation fluid sample at T₁ and by dividing the resistivity of the formation fluid sample at T₃ by the resistivity of the formation fluid sample at T₁. In such embodiments, as further described below, the temperature difference (ΔT) between the first and second temperatures (T₁ and T₂) and the temperature difference (ΔT) between the first and third temperatures (T₁ and T₃) can also be determined.

As noted above, the process 100 can also include determining a salinity of the formation fluid sample 120, based on the resistivity factor value determined in step 115 and the temperature difference (ΔT) between T₁ and T₂. In some embodiments, determining the salinity of the formation fluid sample 120 based on the resistivity factor value and the ΔT can include correlating the resistivity factor value and the ΔT to a pre-generated set of data that can provide a plurality of salinity values at a plurality of resistivity factor values at the ΔT.

In some embodiments, as noted above, once the salinity of the formation fluid has been determined, the process 100 can further include initiating a downhole operation 125 based on the salinity of the formation fluid sample determined in step 120. In some embodiments, downhole operations 125 that can be initiated can be or can include, but are not limited to, carrying out one or more multifunction spectroscopy measurements, one or more nuclear magnetic resonance measurements, one or more dielectric measurements, one or more neutron measurements, one or more sigma measurements, and the like, or any combination thereof. In some embodiments, the multifunction spectroscopy measurement can be carried out with the Pulsar multifunction spectroscopy tool available from Schlumberger. In some embodiments, the sigma measurement can be carried out with the tool disclosed in U.S. Patent Application Publication No. 2010/0187412. Other illustrative downhole operations 125 that can be initiated once the salinity of the formation fluid has been determined can be or can include, but are not limited to, determining and initiating the formation of a new well at a desired location, selecting a particular well depth to initiate hydrocarbon production and initiating hydrocarbon production, determining how to manage and managing produced water, or the like. In other embodiments, once the salinity of the formation fluid has been determined in step 120, the process 100 can further include determining a water saturation of the formation fluid sample based on the determined salinity using any well-known technique. Once the water saturation has been determined, in some embodiments, one or more completion locations that would interface to parts of the formation that are invaded by a relatively high amount of water (as determined from the water saturation) can be avoided, while one or more completion locations that interface to parts of the formation that include a relatively high amount of hydrocarbons (e.g., oil or gas) can be selected as a completion location and completion at the one or more completion locations can be carried out. In other words, completion locations can be selected that include a sufficient amount of hydrocarbons and a sufficiently low amount of water.

In some embodiments, the pre-generated set of data that can be utilized in step 120 can be determined by measuring the resistivity of at least one formation fluid sample that has a known salinity at a plurality of temperatures, e.g., 25° C., 75° C., 100° C., and 135° C., from which a table can be generated that includes the temperatures and the resistivity values at each temperature. In some embodiments, the number of formation fluid samples that have a known salinity can be 1, 2, 3, 4, 5, 6, 7, 8, 9, 10, 11, 12, 13, 14, 15, or more different formation fluid samples, and the resistivity for each formation fluid sample can be measured at the plurality of temperatures or at least at two of the plurality of temperatures, e.g., 25° C. and 135° C. In some embodiments, the number of distinct temperatures the resistivity of the formation fluid having the known salinity can be 2, 3, 4, 5, 6, 7, 8, 9, 10, 11, 12, 13, 14, 15, 16, 17, 18, 19, 20, 21, 22, 23, 24, 25, or more.

In some embodiments, the salinity values for a plurality of known formation fluid samples can preferably be spaced apart from one another in terms of the salinity concentration. For example, when a set of three formation fluid samples having a known salinity concentration are used, the samples can have a salinity concentration of 20 kppm, 100 kppm, and 220 kppm or 10 kppm, 100 kppm, and 250 kppm, or any other desired values.

The first temperature (T₁) the resistivity is measured at, e.g., 25° C., can be referred to as the base temperature measurement. In some embodiments, the resistivity of the formation fluid samples can also be measured at the same base temperature measurement used to generate the pre-generated set of data. For example, when the pre-generated set of data is determined based on a base temperature measurement of 25° C., then the base temperature measurement for each formation fluid sample that has an unknown salinity concentration can also be measured at the same base temperature of 25° C. In other embodiments, the resistivity of the formation fluid samples can be measured at a base temperature that can be different than the base temperature measurement used to generate the pre-generated set of data. For example, when the pre-generated set of data is determined based on a base temperature measurement of 25° C., then the base temperature measurement for each formation fluid sample that has an unknown salinity concentration can be measured at a different base temperature, e.g., 50° C. or 75° C. In a preferred embodiment, the base temperature measurement used for each formation fluid sample that has an unknown salinity concentration can be measured at the same base temperature used to generate the pre-generated set of data.

The example described below would typically be generated by measuring a plurality of formation fluid samples having a known salinity concentration in a lab or other surface location since a temperature of 25° C. is well below actual downhole formation temperatures. Should formation fluid samples be measured in a downhole environment, the base temperature measurement will generally be sufficiently greater than 25° C. Usually, a downhole temperature will be at a minimum in a range from 50° C. to 70° C. and will typically have an upper value of about 175° C. As such, the calculations used to determine the pre-generated set of data discussed below that used a base temperature measurement of 25° C., can be recalculated utilizing a base temperature measurement that would be equal to the base temperature present downhole where the formation fluid sample will be measured to provide a more accurate determination of the salinity, but such recalculation is not necessarily required.

An illustrative example as to how the pre-generated set of data can be determined for a base temperature measurement of 25° C. can be as follows. The resistivity of a formation fluid sample that had a known salinity concentration of 200 kppm was measured at a plurality of different temperatures, i.e., 25° C., 75° C., 100° C., and 135° C. The resistivity of two additional formation fluid samples that had a known salinity concentration of 10 kppm and 80 kppm were also measured at a temperature of 25° C. and 135° C. to generate Tables 1 and 2 below.

TABLE 1

| Temp (° C.) | Resistivity (ohm · m) | Resistivity Factor |
|---|---|---|
| 25 | 2.131 | 1 |
| 75 | 1.066 | 0.50025 |
| 100 | 0.802 | 0.37615 |
| 135 | 0.636 | 0.29832 |

TABLE 2

| Temp | 80 kppm, | 10 kppm |
|---|---|---|
| (° C.) | R (ohm · m) | R(ohm · m) |
| 25 | 3.654 | 19.425 |
| 135 | 1.012 | 5.03 |

The resistivity for each of the three known formation fluid samples was plotted versus temperature to provide three separate resistivity versus temperature curves. Fitted power-law equations for each curve were determined. From the three plots, an equation (equation (II)) can be written as:

$${R}_{t} = a {Temp}^{- b} \qquad ( II )$$

where a is the intercept and b is the slope of the fitted power-law equation for each curve. The intercept (a) and slope (b) in the fitted power-law equations for each curve are shown in Table 3.

TABLE 3

| kppm | a | b |
|---|---|---|
| 200 | 21.4252 | 0.71 |
| 80 | 42.342 | 0.761 |
| 10 | 256.13 | 0.801 |

The intercept (a) and slope (b) were then plotted versus salinity and fitted power-law equations were obtained for each curve. Using the two fitted power-law equations obtained for the two curves, the following equations can be derived (equations (III) and (IV)).

$$a = x {Salinity}^{- y} ; \qquad ( III )$$

$$and$$

$$b = z {Salinity}^{2} - n Salinity + k \qquad ( IV )$$

where x is 1.724E+3, y is −8.346E−1, z is 7.914E−7, n is −6.427E−4, and k is 8.073E−1.Equations II, III, and IV can be used to determine the resistivity for a formation fluid sample having any salinity concentration when the base temperature measurement was 25° C. In this example, the following three tables (Tables 4, 5, and 6) were generated based on the above process that utilized the three formation fluid samples that had a known salinity concentration of 10 kppm, 80 kppm, and 200 kppm and used a base temperature measurement of 25° C.

TABLE 4
Formation Brine Salinity (kppm)

| ΔT (C) | 10 | 20 | 30 | 40 | 50 | 60 | 70 | 80 |
|---|---|---|---|---|---|---|---|---|
| 0 | 1.00000 | 1.00000 | 1.00000 | 1.00000 | 1.00000 | 1.00000 | 1.00000 | 1.00000 |
| 5 | 0.86694 | 0.86755 | 0.86817 | 0.86878 | 0.86940 | 0.87002 | 0.87063 | 0.87125 |
| 10 | 0.76834 | 0.76935 | 0.77036 | 0.77137 | 0.77238 | 0.77339 | 0.77440 | 0.77542 |
| 15 | 0.69205 | 0.69332 | 0.69459 | 0.69586 | 0.69713 | 0.69841 | 0.69968 | 0.70096 |
| 20 | 0.63107 | 0.63251 | 0.63396 | 0.63541 | 0.63687 | 0.63832 | 0.63979 | 0.64125 |
| 25 | 0.58109 | 0.58265 | 0.58423 | 0.58580 | 0.58739 | 0.58897 | 0.59056 | 0.59216 |
| 30 | 0.53929 | 0.54095 | 0.54261 | 0.54427 | 0.54595 | 0.54762 | 0.54931 | 0.55099 |
| 35 | 0.50376 | 0.50548 | 0.50721 | 0.50894 | 0.51067 | 0.51242 | 0.51416 | 0.51592 |
| 40 | 0.47315 | 0.47492 | 0.47668 | 0.47846 | 0.48024 | 0.48203 | 0.48382 | 0.48563 |
| 45 | 0.44647 | 0.44827 | 0.45006 | 0.45187 | 0.45368 | 0.45550 | 0.45733 | 0.45917 |
| 50 | 0.42299 | 0.42480 | 0.42662 | 0.42845 | 0.43028 | 0.43212 | 0.43398 | 0.43583 |
| 55 | 0.40214 | 0.40396 | 0.40580 | 0.40764 | 0.40948 | 0.41134 | 0.41321 | 0.41508 |
| 60 | 0.38349 | 0.38532 | 0.38716 | 0.38901 | 0.39087 | 0.39273 | 0.39460 | 0.39649 |
| 65 | 0.36670 | 0.36854 | 0.37038 | 0.37223 | 0.37409 | 0.37596 | 0.37783 | 0.37972 |
| 70 | 0.35150 | 0.35333 | 0.35517 | 0.35702 | 0.35888 | 0.36075 | 0.36263 | 0.36451 |
| 75 | 0.33766 | 0.33949 | 0.34132 | 0.34317 | 0.34502 | 0.34689 | 0.34876 | 0.35065 |
| 80 | 0.32500 | 0.32682 | 0.32865 | 0.33049 | 0.33234 | 0.33420 | 0.33607 | 0.33795 |
| 85 | 0.31337 | 0.31518 | 0.31701 | 0.31884 | 0.32068 | 0.32254 | 0.32440 | 0.32627 |
| 90 | 0.30265 | 0.30445 | 0.30627 | 0.30809 | 0.30992 | 0.31177 | 0.31363 | 0.31549 |
| 95 | 0.29273 | 0.29452 | 0.29632 | 0.29814 | 0.29996 | 0.30180 | 0.30365 | 0.30550 |
| 100 | 0.28352 | 0.28530 | 0.28709 | 0.28889 | 0.29071 | 0.29253 | 0.29437 | 0.29622 |
| 105 | 0.27494 | 0.27671 | 0.27849 | 0.28028 | 0.28209 | 0.28390 | 0.28573 | 0.28757 |
| 110 | 0.26693 | 0.26869 | 0.27046 | 0.27224 | 0.27403 | 0.27584 | 0.27765 | 0.27948 |

TABLE 5
Formation Brine Salinity (kppm)

| ΔT (C) | 90 | 100 | 110 | 120 | 130 | 140 | 150 | 160 |
|---|---|---|---|---|---|---|---|---|
| 0 | 1.00000 | 1.00000 | 1.00000 | 1.00000 | 1.00000 | 1.00000 | 1.00000 | 1.00000 |
| 5 | 0.87187 | 0.87249 | 0.87311 | 0.87311 | 0.87435 | 0.87497 | 0.87559 | 0.87621 |
| 10 | 0.77643 | 0.77745 | 0.77847 | 0.77847 | 0.78051 | 0.78153 | 0.78255 | 0.78358 |
| 15 | 0.70225 | 0.70353 | 0.70482 | 0.70482 | 0.70740 | 0.70870 | 0.70999 | 0.71129 |
| 20 | 0.64272 | 0.64419 | 0.64566 | 0.64566 | 0.64862 | 0.65011 | 0.65160 | 0.65309 |
| 25 | 0.59376 | 0.59536 | 0.59697 | 0.59697 | 0.60019 | 0.60182 | 0.60344 | 0.60507 |
| 30 | 0.55269 | 0.55438 | 0.55609 | 0.55609 | 0.55951 | 0.56123 | 0.56295 | 0.56468 |
| 35 | 0.51768 | 0.51944 | 0.52122 | 0.52122 | 0.52478 | 0.52657 | 0.52836 | 0.53017 |
| 40 | 0.48743 | 0.48925 | 0.49107 | 0.49107 | 0.49474 | 0.49658 | 0.49843 | 0.50028 |
| 45 | 0.46101 | 0.46286 | 0.46472 | 0.46472 | 0.46846 | 0.47034 | 0.47222 | 0.47412 |
| 50 | 0.43770 | 0.43958 | 0.44146 | 0.44146 | 0.44525 | 0.44715 | 0.44907 | 0.45099 |
| 55 | 0.41696 | 0.41885 | 0.42075 | 0.42075 | 0.42458 | 0.42650 | 0.42844 | 0.43038 |
| 60 | 0.39838 | 0.40028 | 0.40219 | 0.40219 | 0.40604 | 0.40798 | 0.40992 | 0.41188 |
| 65 | 0.38162 | 0.38352 | 0.38544 | 0.38544 | 0.38930 | 0.39124 | 0.39320 | 0.39516 |
| 70 | 0.36641 | 0.36832 | 0.37024 | 0.37024 | 0.37410 | 0.37605 | 0.37801 | 0.37998 |
| 75 | 0.35255 | 0.35445 | 0.35637 | 0.35637 | 0.36023 | 0.36218 | 0.36414 | 0.36611 |
| 80 | 0.33984 | 0.34175 | 0.34366 | 0.34366 | 0.34752 | 0.34946 | 0.35142 | 0.35339 |
| 85 | 0.32816 | 0.33006 | 0.33197 | 0.33197 | 0.33581 | 0.33775 | 0.33971 | 0.34167 |
| 90 | 0.31737 | 0.31926 | 0.32116 | 0.32116 | 0.32500 | 0.32693 | 0.32888 | 0.33084 |
| 95 | 0.30737 | 0.30926 | 0.31115 | 0.31115 | 0.31497 | 0.31690 | 0.31884 | 0.32079 |
| 100 | 0.29808 | 0.29995 | 0.30184 | 0.30184 | 0.30564 | 0.30756 | 0.30949 | 0.31143 |
| 105 | 0.28942 | 0.29128 | 0.29315 | 0.29315 | 0.29694 | 0.29885 | 0.30077 | 0.30271 |
| 110 | 0.28132 | 0.28317 | 0.28503 | 0.28503 | 0.28880 | 0.29070 | 0.29261 | 0.29454 |

TABLE 6
Formation Brine Salinity (kppm)

| ΔT (C) | 170 | 180 | 190 | 200 | 210 | 220 | 230 | 240 | 250 |
|---|---|---|---|---|---|---|---|---|---|
| 0 | 1.00000 | 1.00000 | 1.00000 | 1.00000 | 1.00000 | 1.00000 | 1.00000 | 1.00000 | 1.00000 |
| 5 | 0.87683 | 0.87745 | 0.87807 | 0.87870 | 0.87932 | 0.87994 | 0.88057 | 0.88119 | 0.88182 |
| 10 | 0.78460 | 0.78563 | 0.78666 | 0.78769 | 0.78872 | 0.78976 | 0.79079 | 0.79183 | 0.79286 |
| 15 | 0.71259 | 0.71390 | 0.71520 | 0.71651 | 0.71782 | 0.71914 | 0.72045 | 0.72177 | 0.72309 |
| 20 | 0.65458 | 0.65608 | 0.65758 | 0.65909 | 0.66060 | 0.66211 | 0.66362 | 0.66514 | 0.66667 |
| 25 | 0.60670 | 0.60834 | 0.60998 | 0.61163 | 0.61328 | 0.61494 | 0.61660 | 0.61826 | 0.61993 |
| 30 | 0.56641 | 0.56815 | 0.56990 | 0.57165 | 0.57341 | 0.57517 | 0.57693 | 0.57871 | 0.58048 |
| 35 | 0.53198 | 0.53379 | 0.53561 | 0.53744 | 0.53927 | 0.54111 | 0.54296 | 0.54481 | 0.54667 |
| 40 | 0.50215 | 0.50402 | 0.50589 | 0.50778 | 0.50967 | 0.51157 | 0.51347 | 0.51538 | 0.51730 |
| 45 | 0.47602 | 0.47793 | 0.47985 | 0.48178 | 0.48371 | 0.48565 | 0.48760 | 0.48956 | 0.49152 |
| 50 | 0.45292 | 0.45486 | 0.45681 | 0.45877 | 0.46073 | 0.46271 | 0.46469 | 0.46668 | 0.46868 |
| 55 | 0.43233 | 0.43429 | 0.43626 | 0.43824 | 0.44023 | 0.44223 | 0.44423 | 0.44625 | 0.44827 |
| 60 | 0.41384 | 0.41582 | 0.41780 | 0.41980 | 0.42180 | 0.42381 | 0.42584 | 0.42787 | 0.42991 |
| 65 | 0.39714 | 0.39912 | 0.40111 | 0.40312 | 0.40513 | 0.40716 | 0.40919 | 0.41123 | 0.41329 |
| 70 | 0.38195 | 0.38394 | 0.38594 | 0.38795 | 0.38997 | 0.39200 | 0.39404 | 0.39610 | 0.39816 |
| 75 | 0.36809 | 0.37008 | 0.37208 | 0.37409 | 0.37611 | 0.37815 | 0.38019 | 0.38225 | 0.38432 |
| 80 | 0.35537 | 0.35736 | 0.35936 | 0.36137 | 0.36339 | 0.36542 | 0.36747 | 0.36953 | 0.37160 |
| 85 | 0.34365 | 0.34563 | 0.34763 | 0.34964 | 0.35166 | 0.35369 | 0.35574 | 0.35779 | 0.35986 |
| 90 | 0.33281 | 0.33479 | 0.33678 | 0.33879 | 0.34080 | 0.34283 | 0.34487 | 0.34693 | 0.34899 |
| 95 | 0.32275 | 0.32473 | 0.32671 | 0.32871 | 0.33073 | 0.33275 | 0.33479 | 0.33684 | 0.33890 |
| 100 | 0.31339 | 0.31536 | 0.31734 | 0.31933 | 0.32134 | 0.32336 | 0.32539 | 0.32743 | 0.32949 |
| 105 | 0.30465 | 0.30661 | 0.30859 | 0.31057 | 0.31257 | 0.31458 | 0.31660 | 0.31864 | 0.32069 |
| 110 | 0.29648 | 0.29843 | 0.30039 | 0.30237 | 0.30436 | 0.30636 | 0.30838 | 0.31041 | 0.31245 |

### EXPERIMENTAL EXAMPLE I

The resistivity of a formation fluid sample located within a pay zone in a reservoir was measured and found to have a resistivity of 9.406 ohm·m at 75° C. (T1, ambient reservoir temperature). The formation fluid sample was then heated to 100° C. (T2) and the resistivity was measured again and found to be 5.730 ohm·m. Using Equation I, the resistivity factor was calculated to be (5.730/9.406)=0.6092. Using the temperature difference between the two measurements, i.e., 25° C., and the calculated resistivity factor, one can read from Table 6 that this corresponds to brine salinity between about 180 kppm and 190 kppm.

One can validate the data in Tables 4-6 by comparing the resistivity factor for the formation fluid sample that had the known salinity concentration of 200 kppm. For example, as shown in Table 1, the formation fluid sample that had a salinity concentration of 200 kppm had a resistivity factor value of 0.37615 at a temperature of 100° C., with a temperature difference (ΔT) of 75° C. In looking at Table 6, the predicted salinity concentration would be determined to be about 210 kppm, which was very close to the actual value of 200 kppm.

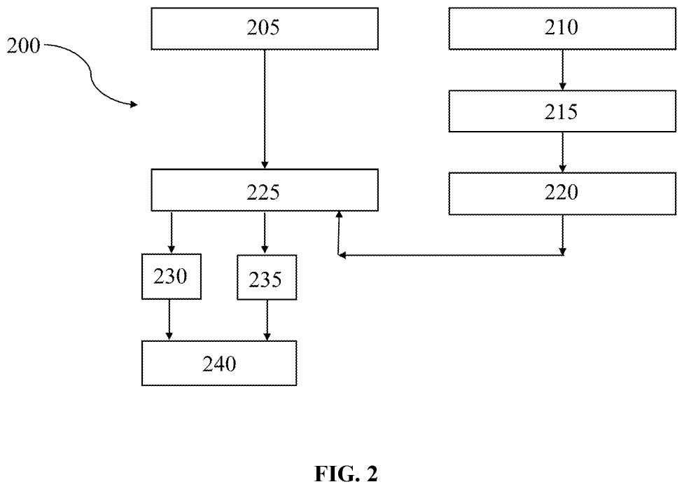

FIG. 2 depicts a flow diagram for an illustrative a process 200 for identifying oil and water bearing zones in a freshwater environment or a low salinity environment, according to one or more embodiments. As used herein, the term “low salinity environment” refers to a formation water that has a salinity concentration of about 200 kppm or less, about 150 kppm or less, about 100 kppm or less, about 75 kppm or less, about 50 kppm or less, about 25 kppm or less, abut 15 kppm or less, or about 10 kppm or less. The process 200 can include generating a resistivity factor value model 205 that can include at least two modeled resistivity factor values at a first temperature (T₁) and a second temperature (T₂), respectively, where T₁ and T₂ can be separated by a temperature difference (ΔT). The process 200 can also include measuring resistivity of a formation fluid sample at the first temperature (T₁) 210. The process 200 can also include measuring resistivity of the formation fluid sample at the second temperature (T₂) 215. The process 200 can also include calculating a resistivity factor value 220 based on the resistivity measured at the first temperature (T₁) and the resistivity measured at the second temperature (T₂). The process 200 can also include determining 225 whether the formation fluid sample is a water-bearing formation fluid sample 230 or an oil-bearing formation fluid sample 235 based on the calculated resistivity factor value obtained in step 220 by comparing the calculated resistivity factor value to a corresponding modeled resistivity factor in the resistivity factor value model 205. The process 200 can also include initiating a downhole operation 240 based on the determination of the formation fluid sample being a water-bearing formation fluid sample 230 or an oil-bearing formation fluid sample 235.

Illustrative downhole operations 240 that can be initiated can be or can include, but are not limited to, carrying out one or more multifunction spectroscopy measurements, one or more nuclear magnetic resonance measurements, one or more dielectric measurements, neutron measurements, one or more sigma measurements, and the like, or any combination thereof. Other illustrative downhole operations 240 that can be initiated once the formation fluid has been determined to be a water-bearing formation fluid 230 or an oil-bearing formation fluid 235 can be or can include, but are not limited to, determining and initiating the formation of a new well at a desired location, selecting a particular well depth to initiate hydrocarbon production and initiating hydrocarbon production, determining how to manage and managing produced water, or the like. In other embodiments, once the formation fluid has been determined to be a water-bearing formation fluid 230 or an oil-bearing formation fluid 235, the process 200 can further include determining hydrocarbon production locations that would interface to parts of the formation that include a relatively high amount of hydrocarbons (e.g., oil or gas) and producing hydrocarbons at such locations while avoiding parts of the formation that are invaded by a relatively high amount of water.

As described above with reference to FIG. 1, in some embodiments, the resistivity of the formation fluid sample at the first and second temperatures (T₁ and T₂) can be measured at the surface, e.g., in a laboratory, mobile analysis unit, or anywhere else. Likewise, as described above with reference to FIG. 1, in other embodiments, the resistivity of the formation fluid sample at the first and second temperatures (T₁ and T₂) can be measured at a downhole location within a wellbore or within the formation the wellbore traverses. Furthermore, as described above with reference to FIG. 1, the resistivity of the formation fluid sample can be measured at the plurality of temperatures while the formation fluid sample remains under the same or substantially the same pressure.

The resistivity factor value model 205 can be generated via any suitable process. In some embodiments, the resistivity factor value model 205 can be generated using a measured water/brine resistivity (R_{w}), a water saturation (S_{w}), a porosity (φ), a saturation exponent (n) and a cementation factor (m) of a rock sample or a formation. Knowing these properties for either the rock sample or the reservoir, using the following equation (V), the formation and fluid resistivity (Rₜ) can be calculated:

$${R}_{t} = \frac{{R}_{w}}{{S}_{w}^{n} {φ}^{m}} . \qquad ( V )$$

To calculate the formation resistivity at different temperatures, the well-known Schlumberger equation by Gen-8 (Schlumberger, 1997, Log Interpretation Charts: Houston, TX (Schlumberger Wireline and Testing), SMP-7006) can be used, where the R_{w} can be corrected to account for the temperature, using equation (VI):

$${R}_{t 2} = {R}_{t 1} [ \frac{{T}_{1} + 21.5}{{T}_{2} + 21.5} ] , \qquad ( VI )$$

where T₁ is the lower temperature and T₂ is the higher temperature, for a reservoir or in the lab for a core or rock sample.

As an example, for an aquifer with 10 kppm salinity brine, a porosity of 18.6%, and saturation exponent and cementation factor of 2, the resistivity to the brine (10 kppm) at 20° C. is 0.628 ohm·m, as shown in Schlumberger, 1997, Log Interpretation Charts: Houston, TX (Schlumberger Wireline and Testing), SMP-7006. Now, using equation VI, the resistivity to the brine at the higher temperatures can be calculated, as shown in Table 7 below. The formation resistivity can then be calculated, at higher temperatures, as only the resistivity to brine will change with the porosity, the saturation, and the exponents, remaining constant. The calculated formation resistivity at the higher temperatures are shown in Table 7.

TABLE 7
Resistivity Calculations at Higher Temperature

| T (° C.) | R_{w} (ohm · m) | Rₜ (ohm · m) |
|---|---|---|
| 20 | 0.628 | 18.158 |
| 25 | 0.561 | 16.206 |
| 75 | 0.27 | 7.809 |
| 100 | 0.215 | 6.202 |
| 135 | 0.167 | 4.815 |
| 200 | 0.118 | 3.402 |
| 300 | 0.081 | 2.344 |
| 400 | 0.062 | 1.788 |

Using the calculated data for the formation resistivity at different temperatures, a resistivity factor value can be calculated, using equation (I):

$$Resistivity Factor = \frac{{Resistivity}_{T 2}}{{Resistivity}_{T 1}} \qquad ( I )$$

where Resistivity_{T2} is the resistivity of the formation fluid sample at the second temperature (T₂), Resistivity_{T1} is the resistivity of the formation fluid sample at the first temperature (T₁), and T₂ is greater than T₁. For example, if one takes the lower temperature point (T₁) as 25° C., the resistivity factor can be calculated via equation (I) for any temperature greater than 25° C., as shown in Table 8 below.

TABLE 8
Modeled Resistivity Factor Values

| T (° C.) | R Factor |
|---|---|
| 25 | 1.000 |
| 75 | 0.482 |
| 100 | 0.383 |
| 135 | 0.297 |
| 200 | 0.210 |
| 300 | 0.145 |
| 400 | 0.110 |

The formation fluid sample can be determined to be a water-bearing formation fluid sample when the calculated resistivity factor value substantially matches the corresponding modeled resistivity factor value. As used herein, the term “substantially matches” means the calculated resistivity factor value is within +/−15% or less of the corresponding modeled resistivity factor value. As such, the formation fluid sample can be determined to be an oil-bearing formation fluid sample when the calculated resistivity factor value deviates, i.e., is greater than +/−15%, as compared to the corresponding modeled resistivity factor value.

As described above with reference to FIG. 1, in some embodiments, the process 200 can also include measuring the resistivity of the formation fluid sample at one or more additional temperatures, e.g., a third temperature (T₃), a fourth temperature (T₄), a fifth temperature (T₅), a sixth temperature (T₆), a seventh temperature (T₇), and so on. Additionally, when the resistivity of the formation fluid sample is measured at three or more different temperatures, the temperature difference between any two sets of adjacent temperatures can be the same or different with respect to one another. For example, when the resistivity of the formation fluid sample is measured at a first temperature (T₁), a second temperature (T₂), and a third temperature (T₃), T₁ can be less than T₂, and T₂ can be less than T₃ and the temperature difference (ΔT_{1/2}) between T₁ and T₂ can be the same or different than the temperature difference (ΔT_{2/3}) between T₂ and T₃.

As also described above with reference to FIG. 1, in some embodiments, the temperature difference (ΔT) between T₁ and T₂ and the temperature difference between any additional/optional temperature measurements, can independently be in a range from 1° C., 3° C., 5° C., 7° C., 10° C., 15° C., 20° C., 25° C., 30° C., 35° C., 40° C., 45°° C., 50° C., 55° C., 60° C., 65° C., 70° C., or 75°° C. to 80° C., 85° C., 90° C., 95° C., 100° C., 105° C., 110° C., 115° C., 120° C., 125° C., 130° C., 135° C., 140° C., 145° C., 150° C., 155° C., or 160° C. In some embodiments, the temperature difference (ΔT) can be at least 1° C., at least 3° C., at least 5° C., at least 7°° C., at least 10° C., at least 15° C., at least 20° C., at least 25° C., at least 30° C., at least 35° C., at least 40° C., at least 45° C., at least 50° C., at least 55° C., at least 60° C., at least 65° C., at least 70° C., or at least 75° C.

As also described above, in some embodiments, the resistivity of the formation fluid sample can be measured at the first temperature (T₁) and then the resistivity of the formation fluid sample at the second temperature (T₂) can be measured. In other embodiments, the resistivity of the formation fluid sample can be measured at the second temperature (T₂) and then the resistivity of the formation fluid sample can be measured at the first temperature (T₁). Preferably, the resistivity of the formation fluid sample is measured at the first temperature (T₁) and then the resistivity is measured at the second temperature (T₂) such that the formation fluid sample can be heated from the first temperature (T₁) to the second temperature (T₂) rather than cooled from the second temperature (T₂) to the first temperature (T₁).

### EXPERIMENTAL EXAMPLE II

An experiment was carried out to validate the concept. A set of samples were prepared and the petrophysical data for each sample was measured. Four limestone samples (Lim 1, Lim 2, Lim 3 and Lim 4) and one dolomite sample (Dol 1) were evaluated. The Lim 1 and Lim 2 samples were first fully saturated with 10 kppm NaCl brine, then only the Lim 2 sample was centrifuged with model oil to an irreducible water saturation of 28.77%. The Lim 3 and Lim 4 samples were fully saturated with 200 kppm NaCl brine, then, only the Lim 4 sample was desaturated to an irreducible water saturation of 28.16%. The Dol 1 sample was fully saturated with 10 kppm NaCl brine.

The samples were separately loaded into a high-pressure high-temperature (HPHT) resistivity cell, where the resistivity was measured at elevated temperatures, i.e., 25° C. (T₁), 75° C. (T₂), 100° C. (T₃), and 135° C. (T₄). The measured resistivity data is shown in Table 9. Equation 1 was used to calculate the resistivity factor for all of the samples at different temperatures, as summarized in Table 9. For these calculations, the resistivity measured at 25° C. (T₁) was used as the base temperature measurement.

TABLE 9
Measured Resistivity and Calculated Resistivity Factor Values

| Sample | T (° C.) | Resistivity (ohm · m) | Resistivity Factor |
|---|---|---|---|
| Lim 1 | 25 | 19.43 | 1.000 |
| (Saturated) | 75 | 9.41 | 0.484 |
| | 100 | 6.73 | 0.346 |
| | 135 | 5.03 | 0.259 |
| Lim 2 | 25 | 340.63 | 1.000 |
| (Desaturated) | 75 | 137.88 | 0.405 |
| | 100 | 96.49 | 0.283 |
| | 135 | 71.94 | 0.211 |
| Lim 3 | 25 | 2.13 | 1.000 |
| (Saturated) | 75 | 1.07 | 0.502 |
| | 100 | 0.80 | 0.376 |
| | 135 | 0.64 | 0.3 |
| Lim 4 | 25 | 32.11 | 1.000 |
| (Desaturated) | 75 | 12.59 | 0.392 |
| | 100 | 9.21 | 0.287 |
| | 135 | 7.01 | 0.218 |
| Dol 1 | 25 | 55.04 | 1.000 |
| (Saturated) | 75 | 26.10 | 0.474 |
| | 100 | 19.44 | 0.353 |
| | 135 | 14.28 | 0.259 |

By comparing the values for the Lim 1, Lim 3, and Dol 1 samples to the modelled resistivity factor values, as shown in Table 10 below, it can be seen that the resistivity factor values for the Lim 1, Lim 3 and Dol 1 samples, which were in the saturated state, substantially match. As such, the data for the Lim 1, Lim 3, and Dol 1 samples indicate a water-bearing zone or formation. It should also be noted that it can be concluded that the rock lithology and salinity do not have a significant affect, which means the proposed process can be lithology independent.

TABLE 10
Saturated Examples vs. Modeled Data

| T (° C.) | Modeled R Factor | Lim 1 | Lim 3 | Dol 1 |
|---|---|---|---|---|
| 25 | 1.000 | 1.000 | 1.000 | 1.000 |
| 75 | 0.482 | 0.484 | 0.502 | 0.474 |
| 100 | 0.383 | 0.346 | 0.376 | 0.353 |
| 135 | 0.297 | 0.259 | 0.300 | 0.259 |

By comparing the values for the Lim 2 and Lim 4 samples to the modelled resistivity factor values, as shown in Table 11 below, it can be seen that the resistivity factor values for the Lim 2 and Lim 4 samples, which were in the desaturated state, deviated by more than 15%. As such, the data for the Lim 2 and Lim 4 samples indicates an oil-bearing zone or formation.

TABLE 11
Desaturated Examples vs. Modeled Data

| T (° C.) | Modeled R Factor | Lim 2 | Lim 4 |
|---|---|---|---|
| 25 | 1.000 | 1.000 | 1.000 |
| 75 | 0.482 | 0.405 | 0.392 |
| 100 | 0.383 | 0.283 | 0.287 |
| 135 | 0.297 | 0.211 | 0.218 |

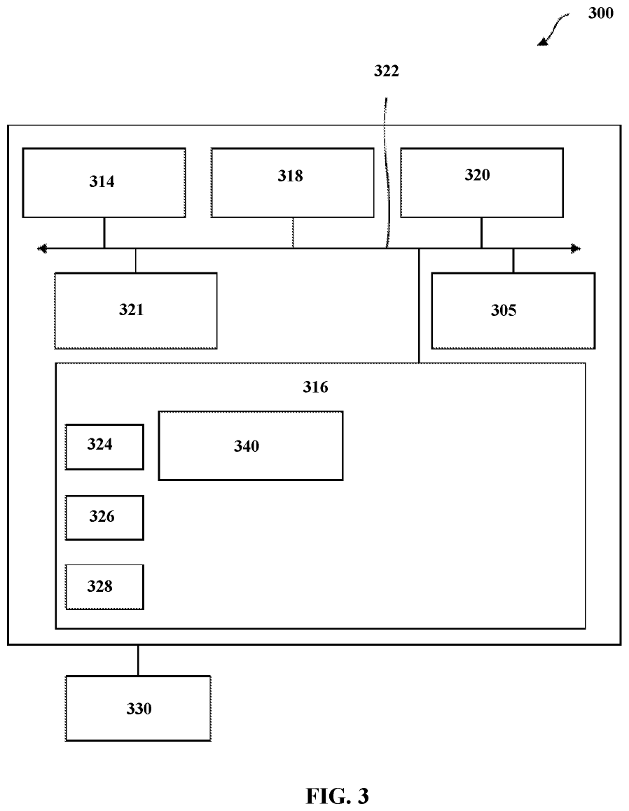

FIG. 3 depicts a schematic of an illustrative computing system 300 that can be configured to carry out one or more steps in the process flow diagrams depicted in FIGS. 1 and/or 2, according to one or more embodiments. The computer system 300 can be located within a facility or can be located elsewhere. One or more chips, for example chips 305 and/or 321, can be or can include field-programmable gate arrays (“FPGAs”), application specific integrated circuits (“ASICs”), chiplets, Multi-Chip-Modules, central processing units (“CPUs”), and/or system-on-chips (“SOCs”), to name a few. The chip can be used in a wide-range of applications, including but not limited to image processing, input data organization, or other digital processing systems. The ASICs can include entire microprocessors, memory blocks including read only memory (ROM), random access memory (RAM), erasable programmable read only memory (EPROM), electrically erasable programmable read only memory (EEPROM), flash memory and other building blocks and can be known as system-on-chip (“SoC”).

To achieve its desired functionality, the computing system 300 can include various hardware and software components. Among these components can be one or more processors 314 and a command actuator 340. These hardware components can be interconnected through the use of a number of electrical connections, buses, and/or network connections. In one embodiment, the processor 314, the chip 305, the chip 321, and the command actuator 340 can be communicatively coupled via a bus 322. The bus 322 can be or include any know computing system bus. The command actuator 340 can be internal to a data storage device 316.

The chip 305, the chip 321, and/or the command actuator 340 can include, either separately or in some combination, software and hardware, including tangible, non-transitory computer readable medium (not shown), for performing reservoir analysis. In some embodiments, the reservoir analysis can be interpreted via statistical formulas, such as average mean, median, standard deviation, and the like, or any combination thereof, and/or complex formulas, such as factor analysis, Fourier's transform, logarithmic methods, and the like, or any combination thereof. Other known algorithms and/or suitable algorithms developed in the future can also be used. In some embodiments, the command actuator 340 can be integrated into the chip 305, the chip 321, and/or the processor 314. In some embodiments, the chip 305 and/or the chip 321 can be integrated into the processor 314. Although the command actuator 340 is depicted as being internal to the data storage device 316, in other embodiments, the command actuator 340 can be a peripheral device (not shown) coupled to the computing system 312 or included within a peripheral device (not shown) coupled to the computing system 312.

The command actuator 340 can include instructions that when executed by the command actuator 340 can cause the command actuator 340 to implement at least the functionality of receiving information through a network adapter, processing the information through the processor according to the instructions stored in the memory to create a command, and for performing reservoir analysis according to the command. In some embodiments, the instructions can, when executed by the command actuator 340, cause the command actuator 340 to use one or more procedures or techniques to perform reservoir analysis using the information received. In some embodiments, the instructions can, when executed by the command actuator 340, cause the command actuator 340 to use one or more analyses to determine the reservoir analysis using the one or more models.

In one or more embodiments, the command actuator 340 can work in conjunction with the processor 314 to implement the functionality described above. In some embodiments, the command actuator 340 can execute firmware code stored on the computing system 300, such as on the chip 305, the chip 321, and/or the processor 314. The functionality of the computing system 300 and/or the command actuator 340 can be in accordance with the processes of the present specification described herein. In the course of executing code, the processor 314 and/or the command actuator 340 can receive input from and provide output to a number of the remaining hardware units.

The computing system 300 can be implemented in an electronic device. Examples of electronic devices include servers, desktop computers, laptop computers, cloud-based computers, personal digital assistants (“PDAs”), mobile devices, smartphones, gaming systems, and tablets, among other electronic devices. The computing system 300 can be utilized in any data processing scenario including, stand-alone hardware, mobile applications, through a computing network, or combinations thereof. Further, the computing system 300 can be used in a computing network, a public cloud network, a private cloud network, a hybrid cloud network, other forms of networks, or combinations thereof. In one example, the processes provided by the computing system 300 can be provided as a service by a third party.

To achieve its desired functionality, the computing system 300 can include various other hardware components. Among these other hardware components can be a number of data storage devices or tangible, non-transitory computer readable medium 316, a number of peripheral device adapters 318, and a number of network adapters 320. These hardware components can be interconnected through the use of a number of electrical connections, busses, and/or network connections.

The chip 305, the chip 321, and/or the processor 314 can include the hardware and/or firmware/software architecture to retrieve executable code from the data storage device 316 and execute the executable code. The executable code can, when executed by the chip 305, the chip 321, and/or the processor 314, cause the chip 305, the chip 321, and/or the processor 314 to implement at least the functionality of receiving information through a network adapter, processing the information from the two or more downhole logs and performing reservoir analysis according to the command.

The data storage device 316 can store data such as executable program code that is executed by the processor 314, the command actuator 340, or other processing devices. The processor 314 can be a central processing unit that is to execute an operating system in the computing system 300. As will be discussed, the data storage device 316 can specifically store computer code representing a number of applications that the processor 314 and/or the command actuator 340 can execute to implement at least the functionality described herein.

In one or more embodiments, the data storage device 316 can include various types of memory modules, including volatile and nonvolatile memory. In one or more embodiments, the data storage device 316 of the present example can include Random Access Memory (“RAM”) 324, Read Only Memory (“ROM”) 326, and Hard Disk Drive (“HDD”) storage 328. Many other types of memory can also be utilized, and the present specification contemplates the use of many varying type(s) of memory in the data storage device 316 as can suit a particular application of the principles described herein. In certain examples, different types of memory in the data storage device 316 can be used for different data storage requirements. In one or more embodiments, in certain examples the processor 314 can boot from Read Only Memory (“ROM”) 326, maintain nonvolatile storage in the Hard Disk Drive (“HDD”) memory 328, and execute program code stored in Random Access Memory (“RAM”) 324. In examples, the chip 305, and the chip 321 can boot from the Read Only Memory (“ROM”) 326.

The data storage device 316 can include a computer readable medium, a computer readable storage medium, or a non-transitory computer readable medium, among others. In one or more embodiments, the data storage device 316 can be an electronic, magnetic, optical, electromagnetic, infrared, or semiconductor system, apparatus, or device, or any suitable combination of the foregoing. More specific examples of the computer readable storage medium can include, for example, the following: an electrical connection having a number of wires, a portable computer diskette, a hard disk, a RAM, a ROM, an EPROM, a Flash memory, a portable compact disc read only memory (“CD-ROM”), an optical storage device, a magnetic storage device, or any suitable combination of the foregoing. In the context of this document, a computer readable storage medium can be any tangible medium that can contain, or store computer usable program code for use by or in connection with an instruction execution system, apparatus, or device. In another example, a computer readable storage medium can be any non-transitory medium that can contain or store a program for use by or in connection with an instruction execution system, apparatus, or device.

The hardware adapters 318, 320 in the computing system 300 can enable the processor 314 to interface with various other hardware components, external and internal to the computing system 300. In one or more embodiments, the peripheral device adapters 318 can provide an interface to input/output devices, such as, for example, a display device 330, a mouse, and/or a keyboard. The peripheral device adapters 318 can also provide access to other external devices such as an external storage device, a number of network devices such as, for example, servers, switches, and routers, client devices, other types of computing devices, and combinations thereof.

The display device 330 can be provided to allow a user of the computing system 300 to interact with and implement the functionality of the computing system 300. Examples of display devices 330 can include a computer screen, a laptop screen, a mobile device screen, a personal digital assistant (“PDA”) screen, and/or a tablet screen, among other display devices 330.

The peripheral device adapters 318 can also create an interface between the processor 314 and the display device 330, a printer, or other media output devices. The network adapter 320 can provide an interface to other computing devices within, for example, a network, thereby enabling the transmission of data between the computing system 300 and other devices located within the network. The network adapter 320 can provide an interface to an external telecommunications network such as a cellular phone network or other radio frequency enabled network, thereby enabling the transmission of data between the computing system 300 and other external devices such as an external storage device, a number of network devices such as, for example, servers, switches, and routers, client servers, radio frequency enabled devices, other client devices, other types of computing devices, and combinations thereof.

The computing system 300 can further include a number of modules used in the implementation of the process and systems described herein. The various modules within the computing system 300 can include executable program code that can be executed separately. In this example, the various modules can be stored as separate computer program products. In another example, the various modules within the computing system 300 can be combined within a number of computer program products; each computer program product including a number of the modules.

The present disclosure further relates to any one or more of the following numbered embodiments:
A1. A process for determining formation water salinity, comprising: measuring resistivity of a formation fluid sample at a first temperature (T₁); measuring resistivity of the formation fluid sample at a second temperature (T₂), wherein T₁ and T₂ are separated by a temperature difference (ΔT); calculating a resistivity factor value based on the resistivity measured at T₁ and the resistivity measured at T₂; determining a salinity of the formation fluid sample based on the resistivity factor value and the ΔT; and initiating a downhole operation based on the determined salinity of the formation fluid sample.
A2. The process of A1, wherein the ΔT between T₁ and T₂ is at least 5° C.
A3. The process of A1 or A2, wherein the ΔT between T₁ and T₂ is in a range from 5° C. to 110° C.
A4. The process of any one of A1 to A3, wherein: the resistivity factor value is calculated according to the equation:

$$resistivity factor value = \frac{resistivity at {T}_{2}}{resistivity at {T}_{1}} ,$$

wherein T₂ is greater than T₁.
A5. The process of any one of A1 to A4, wherein the resistivity of the formation fluid sample measured at T₁ and T₂ is measured downhole.
A6. The process of any one of A1 to A4, wherein the resistivity of the formation fluid sample measured at T₁ and T₂ is measured on a core sample at a surface location.
A7. The process of any one of A1 to A6, wherein determining the salinity of the formation fluid sample based on the resistivity factor value and the ΔT comprises correlating the resistivity factor value and the ΔT to a pre-generated set of data that provides a plurality of salinity values at a plurality of resistivity factor values at the ΔT.
A8. The process of any one of A1 to A7, wherein: the resistivity of the formation fluid sample measured at T₁ is measured before the resistivity of the formation fluid sample is measured at T₂, T₁ is less than T₂, and measuring the resistivity of the formation fluid sample at T₂ comprises heating the formation fluid sample from T₁ to T₂.
A9. The process of A8, wherein the formation fluid sample is heated by directing microwave radiation into the formation fluid sample.
A10. The process of any one of A1 to A9, wherein the downhole operation initiated based on the determined salinity of the formation fluid sample comprises carrying out one or more multifunction spectroscopy measurements, one or more nuclear magnetic resonance measurements, one or more dielectric measurements, neutron measurements, one or more sigma measurements, or a combination thereof.
A11. The process of any one of A1 to A10, wherein the downhole operation initiated based on the determined salinity of the formation fluid sample comprises determining and initiating the formation of a new well at a desired location, selecting a particular well depth to initiate hydrocarbon production and initiating hydrocarbon production, determining how to manage and managing produced water, or any combination thereof.
A12. The process of any one of A1 to A11, further comprising determining a water saturation of the formation fluid sample based on the determined salinity.
A13. The process of A12, wherein the downhole operation comprises selecting one or more completion locations that that interface to parts of a formation that include a relatively high amount of hydrocarbons and carrying out a completion at the one or more selected completion locations.
B1. A process for identifying oil and water bearing zones in a freshwater environment or low salinity environment, comprising: generating a resistivity factor value model comprising at least two modeled resistivity factor values at a first temperature (T₁) and a second temperature (T₂), respectively, wherein T₁ and T₂ are separated by a temperature difference (ΔT); measuring resistivity of a formation fluid sample at T₁; measuring resistivity of the formation fluid sample at T₂; calculating a resistivity factor value based on the resistivity measured at T₁ and the resistivity measured at T₂; determining whether the formation fluid sample is a water-bearing formation fluid sample or an oil-bearing formation fluid sample based on the calculated resistivity factor value by comparing the calculated resistivity factor value to a corresponding modeled resistivity factor in the resistivity factor value model; and initiating a downhole operation based on the determination of the formation fluid sample being a water-bearing formation fluid sample or an oil-bearing formation fluid sample.
B2. The process of B1, wherein the ΔT between T₁ and T₂ is at least 5° C.
B3. The process of B1 or B2, wherein the ΔT between T₁ and T₂ is in a range from 5° C. to 375° C.
B4. The process of any one of B1 to B3, wherein: the resistivity factor value is calculated according to the equation:

$$resistivity factor value = \frac{resistivity at {T}_{2}}{resistivity at {T}_{1}} ,$$

wherein T₂ is greater than T₁.
B5. The process of any one of B1 to B4, wherein the resistivity of the formation fluid sample measured at T₁ and T₂ is measured downhole.
B6. The process of any one of B1 to B4, wherein the resistivity of the formation fluid sample measured at T₁ and T₂ is measured on a core sample at a surface location.
B7. The process of any one of B1 to B5, wherein the resistivity factor value model comprises at least three modeled resistivity factor values at T₁, at T₂, and at a third temperature (T₃), respectively, such that ΔT is equal to ΔT₁, T₂ and T₃ are separated by a temperature difference (ΔT₂), and T₁ and T₃ are separated by a temperature difference (ΔT₃), wherein ΔT₁ and ΔT₂ are equal to one another or different from one another, and wherein ΔT₁ and ΔT₃ are unequal to one another.
B8. The process of B7, further comprising: measuring resistivity of the formation fluid sample at T₃; calculating a resistivity factor value based on the resistivity measured at T₁ and T₃; confirming whether the formation fluid sample is a water-bearing formation fluid sample or an oil-bearing formation fluid sample based on the calculated resistivity factor value at T₃ by comparing the calculated resistivity factor value at T₃ to the modeled resistivity factor value at T₃.
B9. The process of any one of B1 to B8, wherein: the resistivity of the formation fluid sample measured at T₁ is measured before the resistivity of the formation fluid sample is measured at T₂, T₁ is less than T₂, and measuring the resistivity of the formation fluid sample at T₂ comprises heating the formation fluid sample from T₁ to T₂.
B10. The process of B9, wherein the formation fluid sample is heated by directing microwave radiation into the formation fluid sample.
B11. The process of any one of B1 to B10, wherein the downhole operation initiated based on the determination of the formation fluid sample being a water-bearing formation fluid sample or an oil-bearing formation fluid sample comprises carrying out one or more multifunction spectroscopy measurements, one or more nuclear magnetic resonance measurements, one or more dielectric measurements, neutron measurements, one or more sigma measurements, or a combination thereof.
B12. The process of any one of B1 to B11, wherein the downhole operation initiated based on the determination of the formation fluid sample being a water-bearing formation fluid sample or an oil-bearing formation fluid sample comprises determining and initiating the formation of a new well at a desired location, selecting a particular well depth to initiate hydrocarbon production and initiating hydrocarbon production, determining how to manage and managing produced water, or any combination thereof.
B13. The process of any one of B1 to B12, wherein the downhole operation initiated based on the determination of the formation fluid sample being a water-bearing formation fluid sample or an oil-bearing formation fluid sample comprises selecting one or more completion locations that that interface to parts of a formation that include a relatively high amount of hydrocarbons and carrying out a completion at the one or more selected completion locations.

Certain embodiments and features have been described using a set of numerical upper limits and a set of numerical lower limits. It should be appreciated that ranges including the combination of any two values, e.g., the combination of any lower value with any upper value, the combination of any two lower values, and/or the combination of any two upper values are contemplated unless otherwise indicated. Certain lower limits, upper limits and ranges appear in one or more claims below. All numerical values are “about” or “approximately” the indicated value, and take into account experimental error and variations that would be expected by a person having ordinary skill in the art.

Various terms have been defined above. To the extent a term used in a claim can be not defined above, it should be given the broadest definition persons in the pertinent art have given that term as reflected in at least one printed publication or issued patent. Furthermore, all patents, test procedures, and other documents cited in this application are fully incorporated by reference to the extent such disclosure can be not inconsistent with this application and for all jurisdictions in which such incorporation can be permitted.

While certain preferred embodiments of the present invention have been illustrated and described in detail above, it can be apparent that modifications and adaptations thereof will occur to those having ordinary skill in the art. It should be, therefore, expressly understood that such modifications and adaptations may be devised without departing from the basic scope thereof, and the scope thereof can be determined by the claims that follow.

What is claimed is:

1. A process for determining formation water salinity, comprising:
measuring resistivity of a formation fluid sample at a first temperature (T₁);
measuring resistivity of the formation fluid sample at a second temperature (T₂), wherein T₁ and T₂ are separated by a temperature difference (ΔT);
calculating a resistivity factor value based on the resistivity measured at T₁ and the resistivity measured at T₂;
determining a salinity of the formation fluid sample based on the resistivity factor value and the ΔT; and
initiating a downhole operation based on the determined salinity of the formation fluid sample.

2. The process of claim 1, wherein the ΔT between T₁ and T₂ is at least 5° C.

3. The process of claim 1, wherein the ΔT between T₁ and T₂ is in a range from 5° C. to 110° C.

4. The process of claim 1, wherein:
the resistivity factor value is calculated according to the equation:
$$resistivity factor value = \frac{resistivity at {T}_{2}}{resistivity at {T}_{1}} , .$$
wherein T₂ is greater than T₁.

5. The process of claim 1, wherein the resistivity of the formation fluid sample measured at T₁ and T₂ is measured downhole.

6. The process of claim 1, wherein the resistivity of the formation fluid sample measured at T₁ and T₂ is measured on a core sample at a surface location.

7. The process of claim 1, wherein determining the salinity of the formation fluid sample based on the resistivity factor value and the ΔT comprises correlating the resistivity factor value and the ΔT to a pre-generated set of data that provides a plurality of salinity values at a plurality of resistivity factor values at the ΔT.

8. The process of claim 1, wherein:
the resistivity of the formation fluid sample measured at T₁ is measured before the resistivity of the formation fluid sample is measured at T₂,
T₁ is less than T₂, and
measuring the resistivity of the formation fluid sample at T₂ comprises heating the formation fluid sample from T₁ to T₂.

9. The process of claim 8, wherein the formation fluid sample is heated by directing microwave radiation into the formation fluid sample.

10. The process of claim 1, wherein the downhole operation initiated based on the determined salinity of the formation fluid sample comprises carrying out one or more multifunction spectroscopy measurements, one or more nuclear magnetic resonance measurements, one or more dielectric measurements, neutron measurements, one or more sigma measurements, or a combination thereof.

11. A process for identifying oil and water bearing zones in a freshwater environment or low salinity environment, comprising:
generating a resistivity factor value model comprising at least two modeled resistivity factor values at a first temperature (T₁) and a second temperature (T₂), respectively, wherein T₁ and T₂ are separated by a temperature difference (ΔT);
measuring resistivity of a formation fluid sample at T₁;
measuring resistivity of the formation fluid sample at T₂;
calculating a resistivity factor value based on the resistivity measured at T₁ and the resistivity measured at T₂;
determining whether the formation fluid sample is a water-bearing formation fluid sample or an oil-bearing formation fluid sample based on the calculated resistivity factor value by comparing the calculated resistivity factor value to a corresponding modeled resistivity factor in the resistivity factor value model; and
initiating a downhole operation based on the determination of the formation fluid sample being a water-bearing formation fluid sample or an oil-bearing formation fluid sample.

12. The process of claim 11, wherein the ΔT between T₁ and T₂ is at least 5° C.

13. The process of claim 11, wherein the ΔT between T₁ and T₂ is in a range from 5° C. to 375° C.

14. The process of claim 11, wherein:
the resistivity factor value is calculated according to the equation:
$$resistivity factor value = \frac{resistivity at {T}_{2}}{resistivity at {T}_{1}} , .$$
wherein T₂ is greater than T₁.

15. The process of claim 11, wherein the resistivity of the formation fluid sample measured at T₁ and T₂ is measured downhole.

16. The process of claim 11, wherein the resistivity of the formation fluid sample measured at T₁ and T₂ is measured on a core sample at a surface location.

17. The process of claim 11, wherein the resistivity factor value model comprises at least three modeled resistivity factor values at T₁, at T₂, and at a third temperature (T₃), respectively, such that ΔT is equal to ΔT₁, T₂ and T₃ are separated by a temperature difference (ΔT₂), and T₁ and T₃ are separated by a temperature difference (ΔT₃), wherein ΔT₁ and ΔT₂ are equal to one another or different from one another, and wherein ΔT₁ and ΔT₃ are unequal to one another.

18. The process of claim 17, further comprising:
measuring resistivity of the formation fluid sample at T₃;
calculating a resistivity factor value based on the resistivity measured at T₁ and T₃;
confirming whether the formation fluid sample is a water-bearing formation fluid sample or an oil-bearing formation fluid sample based on the calculated resistivity factor value at T₃ by comparing the calculated resistivity factor value at T₃ to the modeled resistivity factor value at T₃.

19. The process of claim 11, wherein:
the resistivity of the formation fluid sample measured at T₁ is measured before the resistivity of the formation fluid sample is measured at T₂,
T₁ is less than T₂, and
measuring the resistivity of the formation fluid sample at T₂ comprises heating the formation fluid sample from T₁ to T₂.

20. The process of claim 11, wherein the downhole operation initiated based on the determination of the formation fluid sample being a water-bearing formation fluid sample or an oil-bearing formation fluid sample comprises carrying out one or more multifunction spectroscopy measurements, one or more nuclear magnetic resonance measurements, one or more dielectric measurements, neutron measurements, one or more sigma measurements, or a combination thereof.
